(19) United States
(12) Patent Application Publication
(10) Pub. No.: US 2015/0190505 A1
(43) Pub. Date: Jul. 9, 2015

(54) LIVE AND IN-VIVO TUMOR SPECIFIC CANCER VACCINE SYSTEM DEVELOPED BY CO-ADMINISTRATION OF EITHER AT LEAST TWO OR ALL THREE OF THE FOLLOWING COMPONENTS SUCH AS TUMOR CELLS, AN ONCOLYTIC VIRUS VECTOR WITH TRANSGENIC EXPRESSION OF GM-CSF AND AN IMMUNE CHECKPOINT MODULATOR

(71) Applicant: Alex Wah Hin YEUNG, (US)

(72) Inventor: Alex Wah Hin Yeung, Newport Beach, CA (US)

(21) Appl. No.: 14/409,383

(22) PCT Filed: Jul. 22, 2013
(86) PCT No.: PCT/US13/51535

§ 371 Date: Dec. 18, 2014

Related U.S. Application Data
(60) Provisional application No. 61/741,804, filed on Jul. 30, 2012.

(51) Int. Cl. A61K 39/395; A61K 39/235

(52) U.S. Cl. CPC A61K 39/39558; A61K 39/235; A61K 2039/505

(57) ABSTRACT

The invention discloses a novel tumor-specific complete vaccine system generated in-vivo. This vaccine system is developed by the use of separated tumor cells inactivated by irradiation and the in-vivo interaction with an oncolytic viral vector with transgenic expression of GM-CSF, completed with immune checkpoint modulators (“ICM”) such as co-stimulatory signals confirmation with an anti-CTLA4 antibody. Specifically there will be no pre-incubation or interaction of the either two or all three components before administration to the patient. One of such oncolytic viral vector examples is CG0070 (GM-CSF expressing conditionally replication competent adenovirus). Mixing of the tumor-viral-ICM components will take place just prior to administration to preserve the effects of the oncolytic process and subsequent immunotherapeutic responses to be live and in vivo from the very first beginning. This invention is a complete live and in-vivo cancer vaccine system (“CLIVS”).

### FIELD OF THE INVENTION

The present invention relates to novel cancer vaccine combinations. In particular, the present invention relates to tumor-specific immunotherapeutic vaccine system comprising irradiated tumor cells, an oncolytic viral expressing GM-CSF, and an immune checkpoint regulatory molecule, kits comprising the tumor-specific immunotherapeutic vaccine system and methods of use therefor.

### BACKGROUND OF THE INVENTION

An oncolytic virus with transgenic expression of GM-CSF, such as CG0070, has been preliminary successfully when given intravesically in bladder cancer [V0046 trial]. Long term complete responses lasting more than a few years have been reported after only a six weekly course of therapy, raising possibilities that this agent is indeed be able to mount a tumor specific immunotherapy against this cancer. To further expand upon this theory, it is proposed here to use a unique way of interaction by a live vaccine system for other cancers that are not easily accessible as those inside the bladder. The first component is the patient's own tumor cells (though allogeneic tumor cells may also be used), taken from biopsy or from a surgical specimen. The second component is an oncolytic virus with GM-CSF expression such as CG0070. The third component is the immune checkpoint modulators, an example given is the co-stimulatory signals confirmation molecule anti-CTLA4 antibody. Instead of pre-incubation or interaction such as in all past methodologies of cancer vaccine to generate antigens or virus attached, infected or modified tumor cells in-vitro, the present novel tumor-virus-ICM vaccine or CLIVS will be developed, live and in-vivo, by mixing the three components only just prior to administration to patients. This will allow the full oncolytic and immunogenic effects to be within the real time reaction of the patient's own immune system. It is believed that such novel method of delivery will enhance the chance of a tumor specific tumor immunotherapy that has never been realized before.

In addition, other immune checkpoint modulators, anti-suppressor cell molecules or stimulation of sustaining co-confirmatory signals modulators can also be co-administered to reinforce strong and long lasting tumor specific immune reactions. Alternatively, immune checkpoint modulators confirmation may be omitted in certain type of cancers.

### SUMMARY OF THE INVENTION

In one aspect of the invention, a tumor-specific immunotherapeutic vaccine system comprising either at least two or all three components: separated tumor cells isolated and inactivated by irradiation, an oncolytic viral and a cancer specific vector comprising a heterologous nucleic acid encoding GM-CSF and an immune checkpoint modulator (“ICM”), wherein the three components are admixed just prior to administration to patient without any pre-incubation are provided.

In another aspect of the invention, the immune checkpoint modulator (ICM) is omitted from the vaccine system.

In certain aspects, the immune checkpoint molecule (ICM) is an anti-CTLA4 antibody. In certain preferred embodiments, the anti-CTLA4 antibody is selected from ipilimumab, tremilimuab and a single chain anti-CTLA-4 antibody.

In other aspects, the ICM is the OX40 binding agent or agonist, or an OX40L molecule that can maintain T cell proliferation beyond the first few days.

In another embodiment, the ICM are antibodies or modulators against PD1, TIM3, B7-H3, B7-H4, LAG-3 and KIR or its ligands.

In another embodiment, the ICM component can be the combination of two or more of ICMs as described in previous embodiments.

In some embodiments, the separate tumor cells are collected and prepared from an autologous, allogeneic or a combination of autologous and allogeneic cells. The autologous and allogeneic cells in certain embodiments may be prepared from cell cultures. In other embodiments, the tumor cells may have been modified to secrete agents that will enhance immune modulation. One of these examples is the GVAX autologous or allogeneic cancer cell therapy, the cells of which secrete GM-CSF.

In another aspect, the oncolytic viral component is from an adenovirus, an example is CG0070, an adenoviral vector with an E2F promoter and transgene expression of GM-CSF.

In another aspect, the oncolytic viral component is any one of the other oncolytic viruses that will be able to express GM-CSF after transduction. Examples, but not exclusively limited to this list, are Herpes Simplex virus, Vaccinia virus, Mumps virus, Reovirus and Newcastle Disease Virus.

In one aspect of the invention, the CLIVS is given subcutaneously to the patient.

In another aspect of the invention, the CLIVS can be given by other routes to the patient, such as epidermal, intramuscular, into lymphatic chains and other sites or organs known to those familiar with the art and that may enhance immune response.

In other aspects of the invention, kits comprising: separated tumor cells isolated and inactivated by irradiation, an oncolytic viral and a cancer specific vector comprising a heterologous nucleic acid encoding GM-CSF and an immune checkpoint modulator (“ICM”), and a packaging insert containing directions for use are provided.

In other aspects of the invention, tumor cell preparation kits comprising: materials and methods to conduct tumor dissociation and preparation, enzymatic and/or virus vector transduction agents, cryopreservation vials etc. and a packaging insert containing directions for use.

### BRIEF DESCRIPTION OF THE DRAWINGS

FIG. 1 is a schematic diagram of CG0070 and wild type (wt) adenovirus type 5. CG0070 is based on adenovirus serotype 5 and the endogenous E1a promoter and E3 19kd) coding region have been replaced by the human E2F-1 promoter and a cDNA coding region of human GM-CSRF, respectively.

FIG. 2.2.1.1 illustrates two bar charts. Selective E1a gene transcription and GM-CSF production in normal Wi38 fibroblasts and Wi38-VA13 cells. A. Selective E1a gene transcription. Wi38 (normal Rb pathway) and Wi38-VA13 (defective Rb pathway) cells were mock infected or infected with CG0070 at 100 or 1000 viral particles (vp) per cell (ppc) for 1 hour on ice followed by incubation at 37° C. to synchronize viral uptake. Quantitative RT-PCR for E1a mRNA was performed 24 hours post infection. E1a RNA levels were normalized to hexon DNA copy number determined at 4 hours post-infection. *p<0.01 t-test, E1a RNA in Wi38-VA13 vs. E1a RNA in Wi38 infected with the same vector. B. Selective GM-DXF production. The supernatants from CG0070-infected Wi38 and Wi38-VA13 cells (100 ppc) were analyzed for human GM-CSF 15 24 hours by ELISA. *p0<0.01, t-test, GM-DSF level in Wi38-VA13 cells vs. Wi38 cells.

FIG. 2.2.2.1 illustrates a bar chart. Productivity of CG0070 and wild type adenovirus in Rb pathway-defective human bladder TCC cells and normal cells. Monolayers of 293, human bladder TCC cell lines (RT4, SW780, UC14 and 253J B-V cells) and human normal cells (fibroblasts MRCS and aortic endothelial hAEC) were infected with either CG0070 or wild type adenovirus at a MOI of 2 plaque forming units (pfu)/cell. Cells were harvested 72 hours after infection and virus titers were determined by plaque assay on 293 cells. The average of duplicate titers from two independent experiments was determined and normalized on 293 cells.

FIG. 2 illustrates a graph. Anti-tumor efficacy of CG0070 in the subcutaneous 253J B0V bladder TCC xenograft model. NCR.nude mice bearing subcutaneous tumors received intratumoral injections of saline or CG0070 five times as indicated by the arrows (SD 1, 3, 5, 8 and 10). The group average tumor volumes ±standard deviation (n=10 per group) are shown for mice that received saline or 3×10⁸ vp, 3×10⁹ or 3×10¹⁰ vp of CG0070 per injection. No significant difference in anti-tumor efficacy was observed among three treatment groups during the course of the study. All of the CG0070-treated groups were all statistically different from the saline treated group (p<0.001, t-test) on SD 60. Between SD 43 and SD 47, 4/10 animals in the PBS group were euthanized due to tumor volume resulting in a drop in the average tumor volume in this group.

FIGS. 2.3.2A-C are reproductions of test mice images. Anti-tumor efficacy of CG0070 in SW780-Luc orthotopic bladder tumor model. Following establishment of orthotopic bladder tumors, mice were treated intravesically with 50 μL of either PBS or CG0070 at the dose indicated in the figure. Mice were imaged every week following intraperitoneal injection of Luciferin. The images shown were taken on SD 1 (A-C) and SD 32 (C) or SD 42 (B) for all animals except as indicated in the figure.

FIG. 2.3.3 illustrates two graphs. Serum GM-CSF expression. CG0070 and Ar20-1004 (2×10¹⁰ vp/injection) were injected intratumorally on SD 1, 3, and 5 as indicated by arrows. Mice were bled and tumors were removed on indicated SD, and serum (panel A) and tumor extracts (panel B) were prepared and assayed for GM-CSF using human- or murine-specific ELISAs. No GM-CSF was detected in mice injected with saline (not shown). Each data point represents the average ±standard deviation of 5 mice.

FIG. 2.3.4 illustrates a single graph. Anti-tumor efficacy in established CMT-64 tumors. CMT-64 tumors were established subcutaneously in C57Bl/6 mice. When the tumors reached an average of 120 mm³, saline or adenovirus (2×10¹⁰ vp) was injected once daily or three consecutive days, as indicated by the arrows. Treatments are indicated in the graph insets. Data represent the average tumor volume and standard error of the mean (n=9 per group). Asterisks indicate p<0.05 versus saline. Asterisks below a symbol indicates significance only for the treatment group directly above the symbol whereas the asterisk above symbols indicates significance for all groups below the symbol compared to saline injection.

FIG. 2.3.5 illustrates a bar chart. Tumor-draining lymph node enrichment of CD11c+ cells in the CMT-64 tumor model following treatment with Ar20-1004. CMT-64 tumor-bearing C57Bl/6 mice were injected daily with HBSS (saline) or 1×10¹⁰ particles/injection of Ar20-1004 or Ar20-1061 for 4 days. Four days after the last injection, mice were euthanized and the tumor-draining lymph nodes (right inguinal lymph node) were collected from each mouse. Single ell suspensions were prepared and stained with an anti-mouse CD11 c monoclonal antibody. Each bar represents the man percent positive cell staining ±SD (n=10 per group). The actual percent positive staining (background subtracted) is indicated above the bars. The asterisk indicated p<0.001 compared to HBSS or Ar20-1061, by one-way ANOVA.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference.

Cancer treatment vaccines, in various combinations and methodologies, have been developed in the past few decades without much clinical success. The human immune system of innate and adaptive immunity is an extremely complex system that has never been successfully utilized to fight against cancer. One explanation is, since cancers are usually developed within the later part of life, the development of an immunological response to counteract cancer is not vital to the survival of the fittest theory in the evolutionary process. In all likelihood, the different aspects of the human immune system are not designed specifically for that purpose.

This invention relates to the preparation of a complete live and in-vivo tumor-virus vaccine system, and its use in immunotherapy of tumor treatment and metastases. This complete live and in-vivo cancer vaccine system (CLIVS) is then able to develop the specific cancer immunotherapeutic effects.

Even after extensive removal of the primary tumor it is still a problem to prevent the formation of metastases either due to growing out of micro-metastases already present at the time of surgery, or to the formation of new metastases by tumor cells or tumor stem cells that have not been removed completely or being re-attached after surgery. In essence, for later stages of cancer, surgery and/or radiotherapy can only take care of the macroscopic lesions, while most patients will have their cancers grow back and not amenable to further therapies.

More recently FDA has approved two immunotherapeutic agents against prostate cancer and melanoma. The first agent, Provenge, utilizes GM-CSF fusion molecule with a prostatic antigen to activate the mononuclear or antigen presenting cells of late stage patients in-vitro and is able to lengthen the overall survival of these patients. The second agent is an anti-CTLA 4 monoclonal antibody that had shown a profound enhancing effect on immune checkpoint modulators signals in T effector cell generation for melanoma patients. An oncolytic virus CG0070 was also been shown to have a long-term complete response effect in bladder cancer patients after one series of six weekly intravesical treatments [V0046 clinical study report].

An object of the present invention is to prepare a complete tumor-viral-ICM live (tumor and viral vector both) in-vivo (vaccine generated in patient) vaccine system with immune checkpoint modulators, such as co-stimulatory signals confirmation by an anti-CTLA4 antibody, which can selectively induce a specific immune response of the patient towards the tumor, and thereby enable a systemic effect against residual primary tumor or in metastatic lesions.

Normally tumor-specific immune T lymphocytes in cancer patients, even when they are present, only occur at low frequency among the lymphocytes. The likely reason is that the antigenicity and immunogenicity of tumor antigens is generally weak, as well as the presence of overwhelming amount of suppressor activities through cytokines and regulatory cells such as Treg etc.

It is now found that the activation of the immune system could be crucially improved by combining different specific immunogenic components. The older concepts of using nonspecific components to booster specific components were found to have little success, as the ability for a human body to generate very specific immunological responses against its own cells (most cancer cells are not immunogenic enough to be different from normal cells) is limited by nature. Such an action derived from non-specific immunological component, even if generated, will be short-lived.

The present invention is to introduce a network of specific immune components that have never been tried in combination before to overcome the natural and complicated suppressing activities of the human body and to produce specific immunotherapeutic effects against its own cancer cells.

The first specific component in the present invention is to use the autologous tumor cells isolated from the resected tumor by mechanical and enzymatical methods. Since cancer cells, particular in metastatic sites, are heterogenous mixtures of different clones of cells undergoing rapid replications and frequent mutations, it is always best to have a specific component that may adapt to these changes while or when they do occur. Autologous tumor cells can be prepared from the original surgical specimen, biopsies or from removal of metastatic lesions later on. One of the advantages of this vaccine is that this component can be changed according to the patient's response and the availability of tumor samples. Thus a tumor-viral live and in-vivo vaccine system generated in the primary tumor phase may be different than the one generated later on, using tumor cells from metastatic sites. The ultimate goal, of course, is to adapt the immunotherapeutic response according to the prevailing tumor types, an advantage that cannot be found in recent development of pathway-targeted therapy or monoclonal antibody-directed therapy.

The second specific component is the live and replicative competent cancer specific oncolytic viral vector. One of such examples is the CG0070, which has a promoter of the E1a early viral gene using the cancer specific E2F group of transcriptional proteins. As recent studies showed, E2F protein is active in most cancer and progenitor cells, and not just only associated with RB pathway deficiencies. This component is different from other tumor-viral vaccines in the past. All of these past cancer vaccines are either non-specific, meaning they cause lysis of normal cells or are not administered as a live viral vaccine system without irradiation.

The third specific component of the present tumor-viral live and in-vivo vaccine system is that the oncolytic virus will be able to generate GM-CSF, the crucial cytokine that enable dendritic cell and other antigen presenting cells to mature and be able to both sample and then to cross-present tumor agents to CD4 cells. Even though antigen presenting cells are involved in non specific immune activities, the availability of suitable and sufficient amounts of GM-CSF in situ will move the direction of the immune response towards a more specific manner, namely in the Th1 and Th17 pathway, if there are sufficient as well as immunogenic tumor antigens present. Few past tumor-viral or tumor-infective agents were based on GM-CSF transgenic expression and even if they did, the vaccines were not delivered with a replicative competent form of the virus.

The fourth and one of the most differentiating components of this tumor-viral live and in-vivo cancer vaccine system is that the generation of the vaccine will be developed in-vivo with immune checkpoint modulators, such as the use of co-stimulatory signals confirmation by an anti-CTLA4 antibody. And that is why the invention is named as a complete vaccine system rather than an already manufactured or in-vitro defined vaccine. This sets the current vaccine apart from the closest patented tumor-viral vaccine as shown in U.S. Pat. No. 5,273,745, whereby the tumor-viral vaccine has to be incubated beforehand in serum free media and then irradiated again to confer non-replication of the viral vector. The viral part of the virally adhered tumor cells will be viewed more or less as an adjuvant, such that it can elicit cytokines, mainly interferon (described in the patent) and be a non-specific component in this immunotherapeutic approach. In this present tumor-viral-ICM system, the viral vector is replicate competent, though limited and specific only to cancer cells or RB defective pathway cells, and this replicative process will elicit the live and in-vivo system that has never been described. The most distinctive features is, as the tumor lysis is happening in vivo, the vaccine is formed from the interaction of these components (tumor and live virus), thereby enabling a stable, sufficient supply of immediate cancer cell death proteins such as tumor associated or tumor specific antigens that are vital to the stimulation of a specific tumor response. In recent studies, it has been shown that not only are these cancer antigens important, but the proteins released from cancer cell death, together with the right cytokine or chemokine environment will make this an ideal situation for the antigen presenting cells, mainly dendritic cell, now primed by the GM-CSF mentioned above, to stimulate a successful and sustaining specific tumor response. The real time happening of these events and the immune checkpoint modulators with co-stimulatory signals confirmation by an anti-CTLA antibody will increase the chance of success in this new invention.

This complete live and in-vivo cancer vaccine system or CLIVS is also obviously different from the intra-tumoral injection of oncolytic viruses with GM-CSF expression. The delivery of oncolytic viruses by the intra-tumoral route has the advantage that no tumor cell preparation in the in-vitro setting is necessary, but has obvious disadvantages such as excessive and uncontrollable leakage of virus vectors such that the dose of the viral vector has to be increased to compensate for such a loss, while there will still be no guarantee such a dose can reach the ideal multiplicity of infectious ratio related to tumor cells. Most likely there will also be a lack of exposure and unpredictable receptor or adherence interaction between tumor cells and the virus vector, disruptive tumor blood supply, difficulty in the access of most visceral tumors and the potential spreading of live tumor cells into other parts of the body during injections. Established tumors are also expected to have increased suppressor cell activities and hostile environment for such therapy. It will also be difficult to calculate the effective dose or to add other effective and supplemental immunotherapeutic agents such as anti-CTLA4 antibodies to intra-tumoral injections because of the inherent messy and unpredictable nature of the procedure. Furthermore, intratumoral injection of replication competent oncolytic viral therapy will not be applicable to the adjuvant setting when immunotherapy will have the best predictable effectiveness by preventing recurrence.

As can be shown in recent studies, IL6 and TGFβ are primarily responsible for the auto-immune process development in a number of experimental animal models. The Th17 pathway and the activation and proliferation of CD4 cells associated in this process is driven by the presence of sufficient and immunogenic antigens, together with the availability of mature antigen presenting cells. In our previous study [V0046], one of the important cytokines associated with tumor response is IL6 (results not shown). In this novel CLIVS approach, the cancer specific oncolytic virus vector expressing GM-CSF (e.g. CG0070) will be responsible, hypothetically, for the purely immunogenic effect enabling the helper T cells pathway shift from a mainly Th1 to a mainly Th17, with the presence of apoptotic tumor cells, its associated or specific antigens and mature antigen presenting cells from GM-CSF on-site expression. It will be the first time that any oncolytic viral vector is utilized only for its immunogenic effect, since the oncolytic effect is not meaningful because the tumor cells have already been irradiated and will not be able to proliferate. The use of the CG0070 immunogenic effect is also novel because it is hypothesized that this effect will be the T helper cell pathway type shift, by Th1 to Th17, and not the usual expected viral immunological effects such as causing a cytokine inflammatory reaction for innate cell killing, meaning more or less as an adjuvant and tumor cell antigens production. As a matter of fact and exactly the opposite of the usual theory of how to use oncolytic viral vectors, it will be beneficial for the patient to develop neutralizing antibodies against the viral vector being used for the above T helper pathway shift, as the immune system will then be focused onto the necessity of eliciting the Th17 cytokines and the necessary T effector cells and/or antibodies generation for the auto-immune cancer therapy.

As a summary, the novelty of the present live and in-vivo tumor specific cancer vaccine system is based on the following facts.

Firstly, the tumor and viral components are completely separated until the moment of administration to the patient. No pre-incubation or mixture of these components for any measurable amount of time before treatment. All past cancer vaccines consisting of tumor cells and virus vectors have all involved in-vitro manipulation.

The viral component is cancer specific and replicative competent.

The transgenic expression of GM-CSF is strategically happening at the tumor lytic site.

The generation of the vaccine is live and in-vivo within the patient's body, capturing all the necessary cellular, cytokine and chemokine and antigenic components during cancer cell death to be sampled and cross-presented by the GM-CSF primed and matured antigen presenting or dendritic cells and the immune checkpoint modulators signal confirmation by the anti-CTLA4 antibody.

The easy and dependable access of producing tumor-viral-ICM interactions in the proposed complete live and in-vivo cancer vaccine system to most cancer patients is also immensely different from the setting of intra-tumoral injection of virus vectors.

The administration of the CLIVS approach will also be novel since this will be the first time that a cancer vaccine may be given, with parts of or with all of its components, in multiple doses at the same or different injection sites, by more than once per week. The normal administration schedule of the CLIVS system will likely be an intradermal or subcutaneous injection of all or parts of its vaccine components once per week as a cycle and then the same cycle being repeated every two to three weeks for four (4) to six (6) cycles as one course. To further enhance and sustain the cancer specific immune response, the CLIVS system may also be administered twice or more per week as a cycle, and/or with injections of the vaccine at the same or different physical sites during each cycle. This “tandem” or two doses per week cycle will then be repeated every two to three weeks for a total of four (4) to six (6) cycles as a complete course of treatment.

Finally, yet another aspect of novelty of the system, the why and how to implement the system, is based on the theory that the cancer specific oncolytic virus used is purely to act as an immunogenic agent (not for its oncolytic effect like most being developed are). That immunogenic agent effect is also novel in that it is aimed at a T helper cell type shift, and not as an adjuvant or tumor antigens production like in the past.

### Methods:

### Tumor Cells:

### Preparation of Tumor Cells:

For the usual surgical specimen, a piece of the tumor is removed for pathological classification and the main tumor cell mass is then placed into a tube with HBSS containing gentamycin and stored at 8° C. Within about 8-12 hours, the fresh tumor specimens are carried to the laboratory, where they are further dissociated. The tumor specimens are cut into smaller pieces, usually in 1 cm cubes with a scalpel. They are then incubated in an enzyme solution at 37° C. The usual enzymatic solution most effective is a mixture of collagenase, DNase, and hyaluronidase. After incubation the resulting suspension is filtered through a nylon mesh with a pore of 40 μm. These steps are repeated until all the main fraction of the tumor specimen has been dissolved. The resulting cell suspension is then washed three times in HBSS and then ready for cryopreservation.

### Cryopreservation and Thawing of Tumor Cells:

Tumor cells isolated in this manner are then frozen in 10% human serum albumin and 10% DMSO and stored in aliquots of 107 cells in liquid nitrogen. Cell freezing can be performed in a freezing computer Kryo 10 series II (Messer-Griesheim). On the day of the planned vaccination, the cells are carefully thawed in warm medium with the addition of 10% human serum albumin and then washed three times in this medium.

### Inactivation of the Tumor Cells:

The tumor cells proliferative capacity is inactivated with 200Gy using a telecobalt source prior to administration.

### Viral Vector:

Preparation of an Oncolytic and Cancer Specific Viral Vector with Transgenic Expression of GM-CSF:

While there are many viral vectors that are cancer specific and conditional replicative competent, they are usually classified as either promoter based, attenuated based or genetic manipulation based. The example given here is promoter based CG0070, an adenovirus serotype 5 which has an E2F promoter at the E1a gene and a GM-CSF expression at the E3 gene.

### 1. Physical, Chemical, and Pharmaceutical Properties and Formulation

### 1.1 CG0070

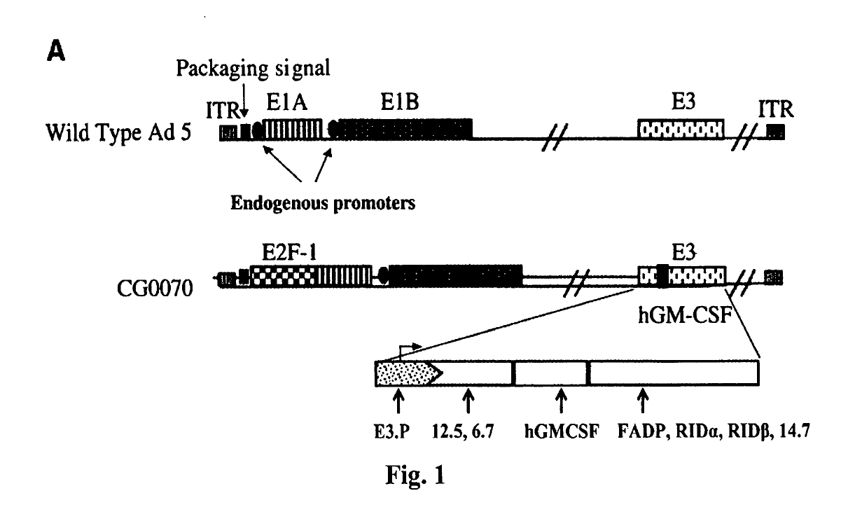

CG0070 is a conditionally replicating oncolytic adenovirus (serotype 5) designed to preferentially replicate in and kill Rb pathway-defective cancer cells. In approximately 85% of all cancers, this pathway is mutated. The genomic structure of the oncolytic adenoviral vector CG0070 is shown schematically in FIG. 1. The human E2F-1 promoter, which provides tumor specificity to any downstream gene products, was cloned in place of the endogenous E1A promoter in the human Ad5 backbone. To protect from transcriptional read-through activating E1A expression, a polyadenylation signal (PA) was inserted 5′ of the E2F-1 promoter. CG0070 includes the entire wild type E3 region except for the 19kD-coding region. A direct comparison of E3-containing to E3-deleted oncolytic adenovirus vectors showed superiority of E3-containing vectors in tumor spread and efficacy. In place of the 19kD gene, CG0070 carries the cDNA for human GM-CSF under the control of the E3 promoter (E3P). The rest of the viral vector backbone, including the E2, E4, late protein regions and inverted terminal repeats (ITRs), is identical to the wild type Ad5 genome.

Products of the adenoviral early E1A gene are essential for efficient expression of other regions of the adenoviral genome. CG0070 has been engineered to express the E1A gene under control of the human E2F-1 promoter, while the expression of GM-CSF is controlled by the endogenous viral E3 promoter. Since the E3 promoter is in turn activated by E1A, both viral replication and GM-CSF expression are ultimately under the control of the E2F-1 promoter. Due to its tumor-selective E2F-1 promoter, CG0070 is designed to replicate in and selectively kill tumor cells with Rb-pathway defects.

Infected cells produce GM-CSF, which is expected to stimulate immune responses against uninfected distant and local tumor foci.

### 1.2 Clinical Production of CG0070 by Cold Genesys, Inc.

CG0070 is manufactured in HeLa-S3 cells, and released from infected HeLa-S3 cells by detergent lysis. CG0070 is purified from the lysate by chromatography, then formulated in 5% sucrose, 10 mM Tris, 0.05% polysorbate-80, 1% glycine, 1 mM magnesium chloride, pH 7.8.

CG0070 is supplied as a sterile, slightly opalescent, frozen liquid in stoppered glass vials. The particle concentration per mL (vp/mL) is stated on the Certificate of Analysis for each lot of CG0070.

### 2. Nonclinical Pharmacology and Toxicology:

### 2.1 Rationale for the Testing Plan

CG0070 is a conditionally replicating oncolytic adenovirus designed to preferentially replicate in and kill Rb pathway-defective cancer cells. The gene for the tumor suppressor RB, or one of the components of its regulatory pathway, is mutated in approximately 60% of all cancers. CG0070 has additional potential anti-tumor activity in that it carries the cDNA for human GM-CSF, a key cytokine for generating long-lasting anti-tumor immunity. Thus, CG0070 is a selectively replicating oncolytic vector with the potential for attacking the tumor by two mechanisms: direct cytotoxicity as a replicating vector and induction of a host immune response. Summarized in the following sections are in vitro and in vivo studies conducted to characterize the tumor selectivity and anti-tumor activity and safety of CG0070.

### 2.2 In Vitro Tumor Selectivity and Cytotoxicity

A series of studies conducted in vitro to characterize the selectivity of CG0070 in normal and tumor cells, including selective gene expression, cytotoxicity, and viral replication in Rb pathway-defective cells are summarized below.

### 2.2.1 Rb-Dependent Expression of E1A and GM-CSF

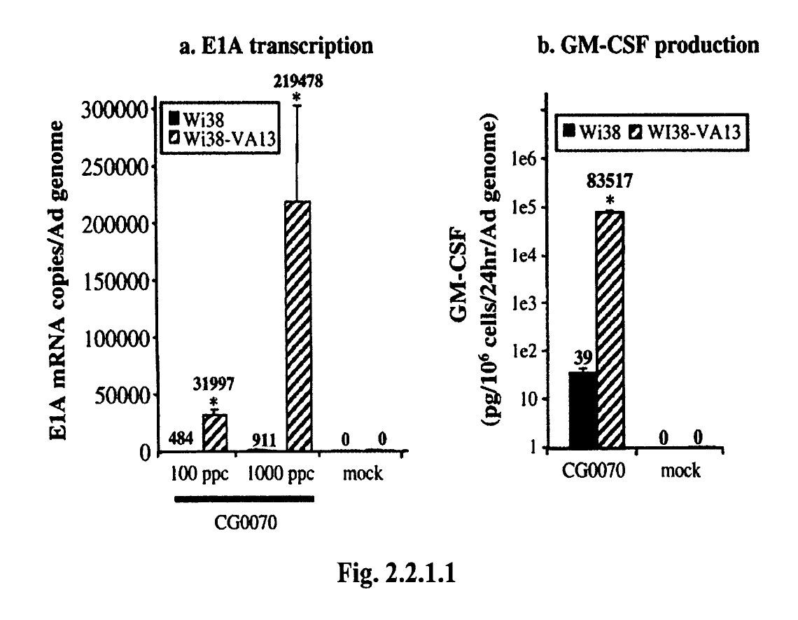

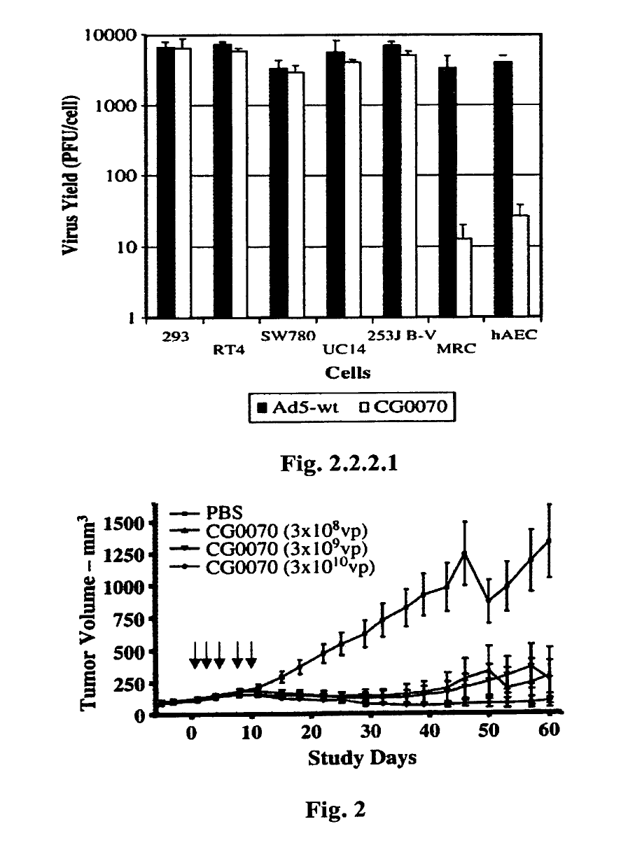

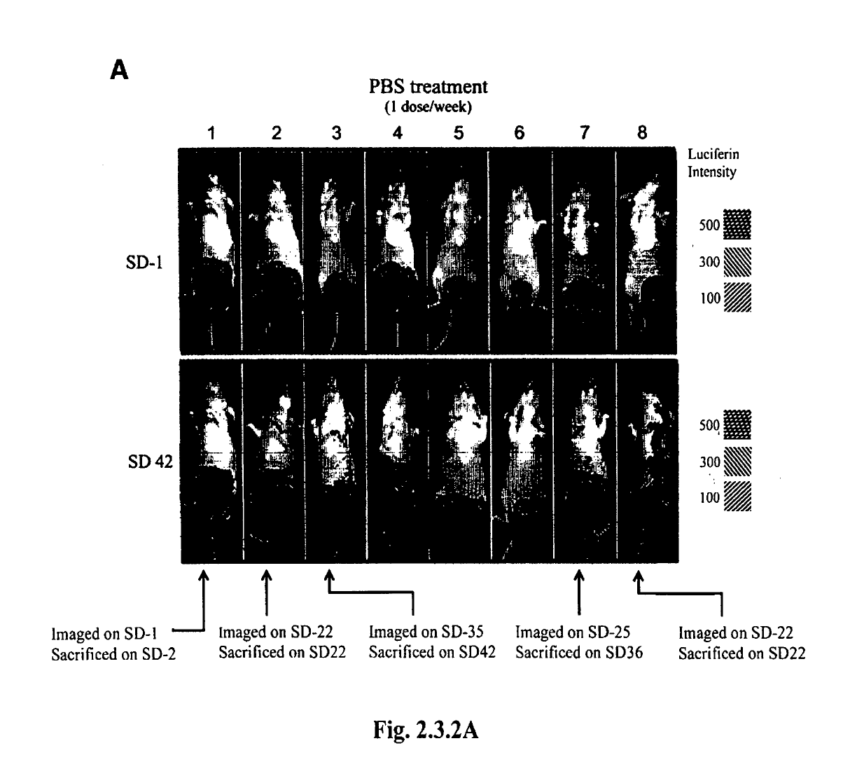

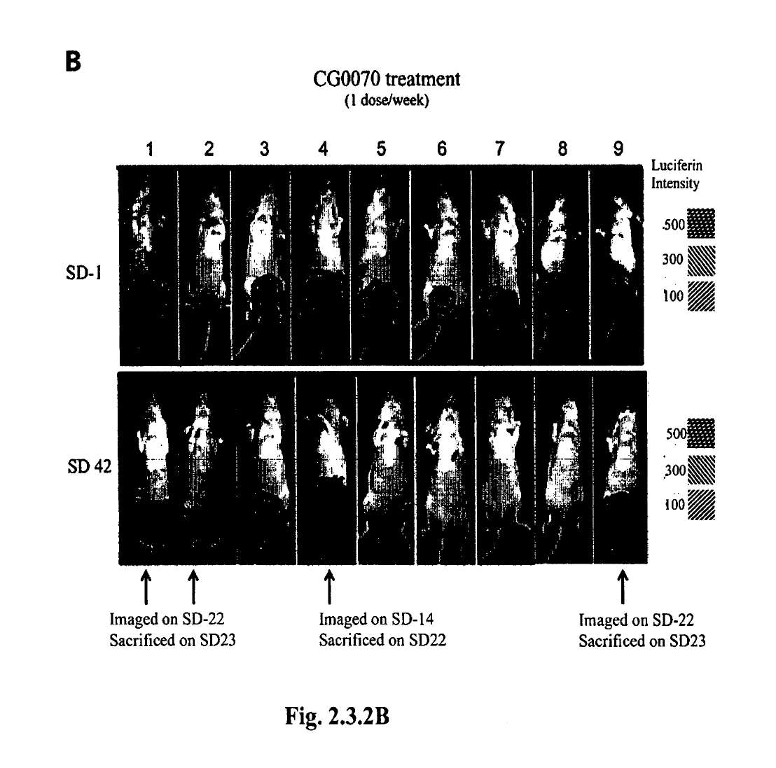

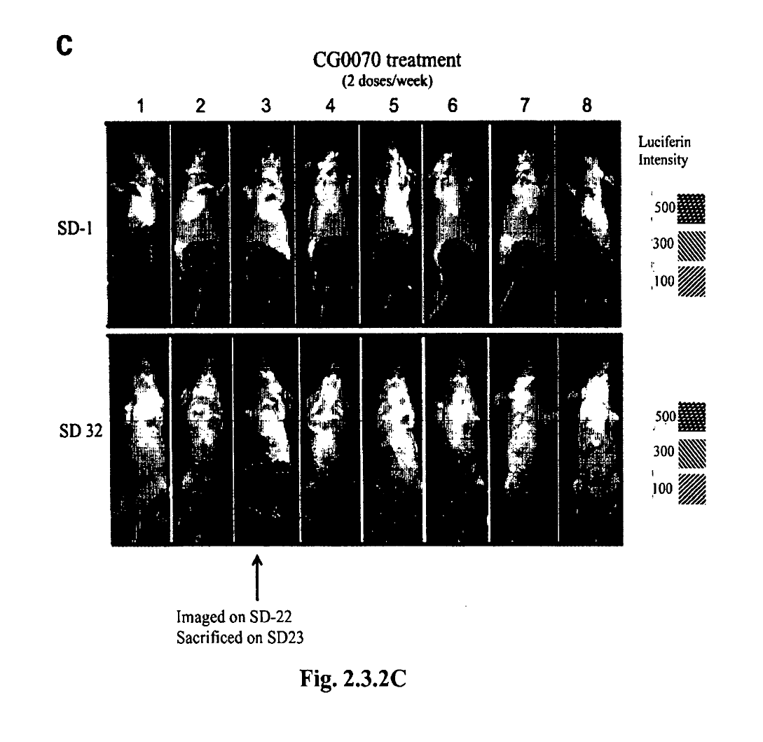

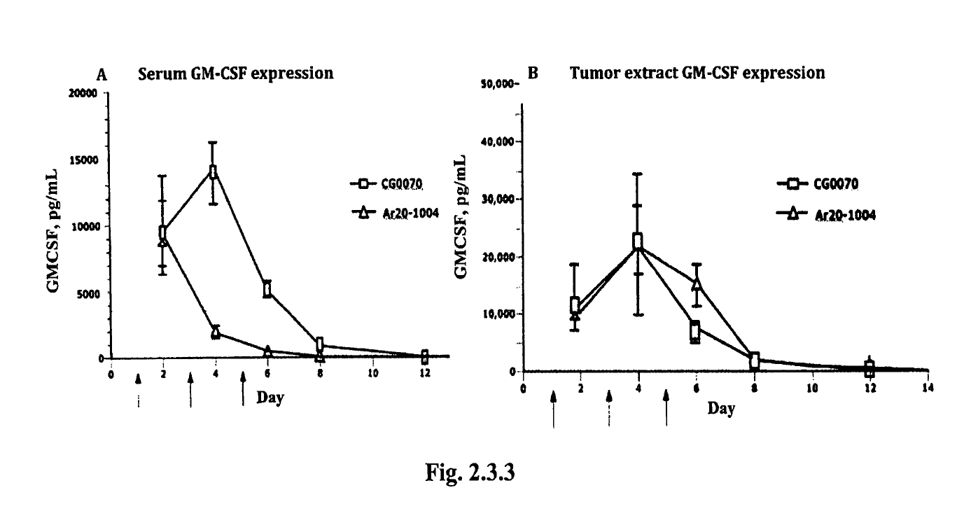

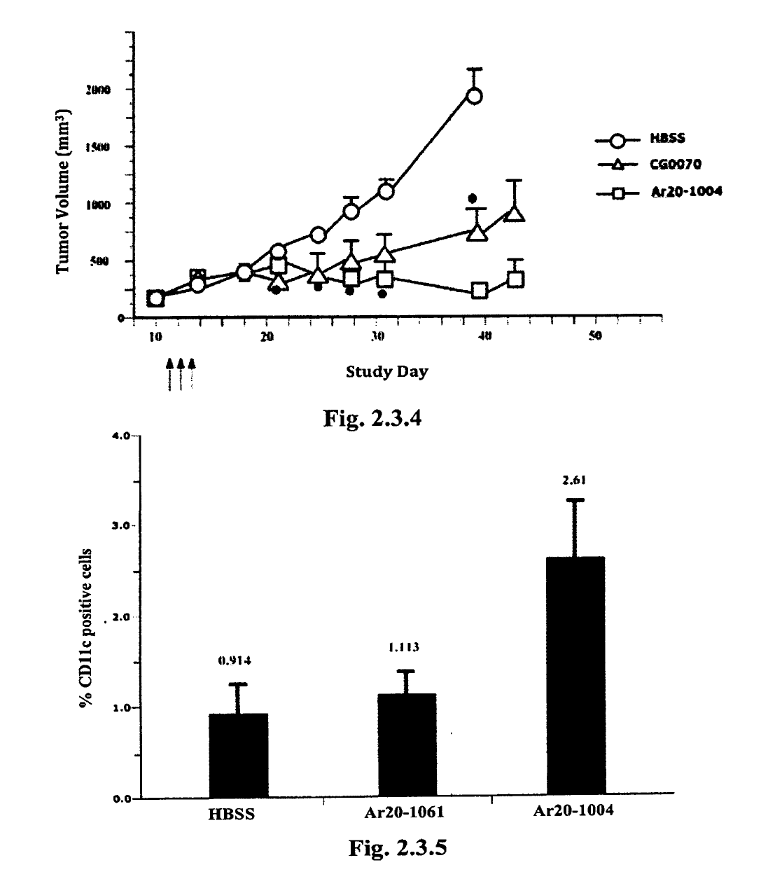

The matched cell lines, Wi38 and Wi38-VA13, were compared for the ability to support CG0070 replication as evidenced by expression of E1a and GM-CSF. Wi38-VA13 is an Rb pathway-deregulated cell line due to constitutive SV40 large T antigen expression. Previously published data confirmed that E2F-1 expression is up-regulated in Wi38-VA13 (Jakubczak et al., 2003). The parental Wi38 cell line is a normal human diploid fibroblast cell line and is not Rb-pathway defective (e.g., Rb wild type). Cell cultures were infected as described in the legend of FIG. 2.2.1.1, and E1a expression was assessed by quantitative RT-PCR (qRT-PCR) 4 hours after inoculation. Data were normalized to the number of Ad genome copies as determined by hexon qPCR. The level of E1a mRNA was significantly higher in the Rb pathway-defective Wi38-VA13 cells than in normal Wi38 cells (FIG. 2.2.1.1). The dependence of GM-CSF production on a defective Rb pathway was also demonstrated, providing evidence that the human E2F-1 promoter in CG0070 is capable of selectively regulating adenoviral E1a gene transcription and downstream E3 promoter-regulated hGM-CSF expression in Rb pathway-defective cells.

### 2.2.2 Selective Production and Cytotoxicity of CG0070 in Rb-Pathway Defective Tumor Cells

Measuring virion production in cells with normal and defective Rb pathway function provides further evidence of the selectivity of CG0070 replication and cytotoxicity. The amount of virus produced reflects numerous processes, including the ability of a particular cell type to be infected, to transactivate viral promoters, and to allow replication of the virus, and thus provides a good measure of the selectivity of a targeted adenovirus. CG0070 replicated in the Rb pathway-defective human bladder TCC cell lines RT-4, SW780, UC-14, and 253J B-V as efficiently as wild type adenovirus, producing similar levels of progeny virus (FIG. 2.2.2.1). CG0070 replicated inefficiently, however, in normal Rb pathway cells.

The cytotoxicity of CG0070 is also dependent upon a defective Rb pathway. A panel of human Rb pathway-defective tumor and normal (non-tumor) cells was infected with CG0070. Based on a quantitative MTS cytotoxicity assay, CG0070 was selectively cytotoxic in Rb pathway-defective tumor cells (Table 2.2.2.1) in which EC5o values were consistently lower than in primary cells.

TABLE 2.2.2.1
Cytotoxicity of CG0070 and wild type Ad5 in human tumor cell lines and primary
cells by MTS assay. The EC₅₀ values (vp/cell resulting in 50% reduction in cell viability) were
calculated by linear regression analysis using a sigmoidal dose-response curve fit. A low EC₅₀
value indicates a stronger cytotoxic agent. The number of replicates for each cell type is listed in
parentheses. Data represent the mean ± standard deviation of the replicates.

| | | | EC₅₀ (mean ± SD) (ppc/cell) | |
|---|---|---|---|---|
| Cells (replicates) | Rb pathway | Cell Type | CG0070 | Wild type Ad5 |

Tumor cell lines

| | | | | |
|---|---|---|---|---|
| UC14 (6) | Defective | Bladder TCC | 0.40 ± 0.29 | 0.015 ± 0.008 |
| RT4 (6) | Defective | Bladder TCC | 1.04 ± 0.96 | 0.03 ± 0.02 |
| SW780 (6) | Defective | Bladder TCC | 3.01 ± 0.25 | 0.17 ± 0.102 |
| 253JB-V (6) | Defective | Bladder TCC | 19.21 ± 0.54 | 4.10 ± 0.47 |
| Hep3B (9) | Defective | Hepatocellular carcinoma | 0.022 ± 0.022 | 0.006 ± 0.005 |
| SW620 (9) | Defective | Colon carcinoma | 4.7 ± 3.7 | 2.9 ± 1.8 |
| LNCaP-C4-2 (6) | Defective | Prostate carcinoma | 1.7 ± 1.2 | 7.7 ± 5.9 |
| PC3M-2Ac6 (9) | Defective | Prostate carcinoma | 227 ± 152 | 65 ± 49 |
| Wi38VA13 (6) | Defective | SV-40-transformed fibroblasts | 30 ± 8 | 4 ± 3 |

Non-tumor cells

| | | | | |
|---|---|---|---|---|
| hMEC (2) | Normal | Mammary epithelial cells | 504 ± 124 | 90 ± 7 |
| hREC (6) | Normal | Renal endothelial cells | 1158 ± 703 | 371 ± 83 |
| hUVEC (6) | Normal | Umbilical vein endothelial cells | 417 ± 195 | 1 ± 0 |
| hMVEC (3) | Normal | Microvascular endothelial cells | 1247 ± 257 | 17 ± 5 |
| NHLF (6) | Normal | Lung fibroblasts | 108 ± 66 | 38 ± 8 |
| MRC5 (6) | Normal | Lung fibroblasts | 127 ± 60 | 5.0 ± 3 |
| W138 (5) | Normal | Lung fibroblasts | 1712 ± 209 | 865 ± 901 |

### 2.2.3 Production of Biologically Active GM-CSF

The production of biologically active GM-CSF after infection with CG0070 is also selective for Rb pathway-defective tumor cells (Table 2.2.3.1). The level and activity of GM-CSF in supernatants derived from human bladder TCC 253J B-V, SW780, RT4 and UC14 cells that were infected with CG0070 at various multiplicities of infection (moi, vp/cell) were measured by ELISA and by cell proliferation assay using the GM-CSF dependent TF-1 erythroleukemia cell line, respectively. At 100 vp/cell or higher moi, production of biologically active GM-CSF exceeded 40 ng/mL/106 cells/24 hr in all cell lines, a level that Dranoff, et al. [Dranoff] have reported is sufficient to induce potent, long lasting anti-tumor immunity in in vivo tumor vaccination models. Therefore, CG0070 has the potential to produce GM-CSF in quantities predicted to be therapeutic.

TABLE 2.2.3.1
Production of biologically active GM-CSF in CG0070-infected bladder TCC cells.
Duplicate wells of human bladder TCC cell lines were infected at the indicated particles/cell
ratios for 24 hours. Cell supernatants were collected and tested for total GM-CSF protein by
ELISA (in duplicate) and for GM-CSF activity using a proliferation bioassay in triplicate on TF-1
erythroleukemia cells. ELISA data represent the mean ± standard deviation of replicate wells.

| | | ELISA | Bioassay |
|---|---|---|---|
| Cell Line | Particles/cell | (ng/10⁶ cells/24 hrs) | (IU/ng) |
| RT4 | 1000 | 1457 ± 60 | 0.68 |
| | 100 | 361 ± 8 | 0.68 |
| | 10 | 40 ± 2 | 0.60 |
| UC-14 | 1000 | 2807 ± 145 | 0.69 |
| | 100 | 992 ± 11 | 0.47 |
| | 10 | 114 ± 3 | 0.56 |
| SW780 | 1000 | 3853 ± 245 | 0.72 |
| | 100 | 707 ± 16 | 0.91 |
| | 10 | 214 ± 7 | 0.81 |
| 253J B-V | 1000 | 1492 ± 101 | 1.33 |
| | 100 | 934 ± 67 | 1.46 |
| | 10 | 153 ± 2 | 1.14 |

### 2.2.4 Summary of In Vitro Studies

CG0070 efficiently replicates in and lyses human tumor cells of various types, including bladder TCC, in vitro and has marked selectivity for tumor cells compared to non-tumor cells. Production of biologically active GM-CSF was induced in a dose-related fashion at levels known to stimulate anti-tumor protective immunity in in vivo tumor models.

### 2.3 Anti-Tumor Activity of CG0070 in Animal Models

The anti-tumor effects of oncolytic vectors like CG0070 can be evaluated in mouse models using human tumor xenografts to assess the inhibitory effect of virus on tumor growth, since the virus will replicate in the human cells. The major mechanism of prevention of tumor growth in these animals is the oncolytic effect of the virus and the direct cytotoxicity of the innate immune system (Natural Killer cells, macrophages, neutrophils, etc.) against the tumor. The limitations of these models are the inability of adenovirus to replicate in murine tissues, limiting the assessment of the replication effects of the virus in the animal, as well as the absence of the T and B cell arms of the immune system, which limits the evaluation of the induction of tumor-specific T cell immunity. Virus replication and the anti-tumor activity of CG0070 have been evaluated after single or multiple intratumoral injections in several different tumor xenograft models, including an orthotopic bladder cancer model in mice. An evaluation of the anti-tumor efficacy of CG0070 in an immunocompetent murine tumor model that can be infected by CG0070 and is able to produce low levels of progeny adenoviral particles is also discussed below.

The anti-tumor activity of CG0070 was evaluated in a subcutaneously implanted bladder TCC 253J B-V xenograft tumor model in NCR nude mice. Because CG0070 expresses human GM-CSF, which is inactive in mice, this study effectively measured only the cytotoxic anti-tumor effects of the virus itself, not the effects of GM-CSF-associated stimulation of the immune system (which cannot readily be measured in immune deficient mice under any circumstances). CG0070 (3×108, 3×109 or 3×1010 vp/dose) or saline was injected intratumorally into subcutaneous 253J B-V bladder tumor xenografts in female mice (n=10 per group). Each animal received five intratumoral injections, on an approximately every other day schedule, beginning when the tumors reached approximately 125 mm3 in size. Tumor volume was measured twice weekly. Anti-tumor activity was observed at all dose levels compared to saline-treated tumors (FIG. 2 below, p<0.001). By SD 60, the saline-treated tumors had increased nearly 16-fold in size, whereas the growth of tumors treated with 3×1010 vp/dose of CG0070 was completely inhibited. Tumor size in the low and mid-dose groups also remained significantly smaller than in the saline treatment group, increasing only by approximately 3.5- to 4-fold by SD 60.

The anti-tumor activity of CG0070 was also examined in an orthotopic bladder tumor model in nude mice. Orthotopic animal models of bladder cancer are predicted to more closely mimic the site and behavior of these tumors in humans. To visualize the tumor growth in living animals, the human bladder TCC cell line SW780-luc, which constitutively expresses luciferase, was used for model development. SW780-Luc cells were intravesically instilled into the bladders of nude mice and the established orthotopic tumors that formed were visualized in vivo by luminescence imaging following intraperitoneal injection of Luciferin, to monitor tumor growth in situ. Immunohistochemical (IHC) analyses of the orthotopic tumor-bearing bladder with human anti-cytokeratin staining confirmed the human origin of the tumors. Histologically, the SW780-Luc orthotopic tumors closely resembled superficial bladder tumors in humans [Ramesh et al., 2004a], confirming the ability of this model to approximate the clinical setting. Earlier studies had demonstrated the need for pretreatment with DDM to enhance infectivity of bladder epithelium with adenovirus [Ramesh et al., 20041)] (see below). A similar DDM pretreatment regimen was found to be essential for efficient transduction of orthotopic tumors by adenovirus.

In the efficacy study, female NCR nude mice bearing orthotopic SW780-Luc bladder tumors received six intravesical doses of CG0070 (3×1010 vp/dose in a 50 μL dosing volume) either once or twice weekly for 3 consecutive weeks. Treatment was initiated 9 days following the tumor cell implantation when the average bioluminescence was approximately 3 to 8×106 photon counts; bladders were pretreated with DDM prior to each virus treatment. Based on tumor imaging in situ (FIG. 2.3.2C), 5/8 animals in the CG0070 treatment group (2 doses/week) were tumor-free by SD 32 following the initiation of treatment. The tumor of one animal had decreased in size (drop from 1.45×106 to 5.71×105 photon count), that of another was stable, and one animal was euthanized with a large tumor burden. In another CG0070 treatment group (1 dose/week), in situ tumor imaging showed that 4/9 animals were tumor-free by SD 42 following the initiation of treatment (FIG. 2.3.2B). The tumor in one animal increased in size, 3 animals were euthanized with a large tumor burden, and one animal died in the cage due presumably to the large tumor as recorded by prior imaging data. In contrast, all of the tumors in the saline treatment group (n=8) increased in size compared to the baseline except for one animal in which the tumor did not grow (FIG. 2.3.2A). Immunohistochemical evaluation of the bladder confirmed the complete absence of tumor cells in CG0070-treated mice (5/8 in 2 doses/week group and 4/9 in 1 dose/week group) deemed tumor free by in vivo imaging.

The assessment of the immune-enhancing effect of GM-CSF expressed by cells infected with a replicating adenovirus in murine tumor models is constrained because adenovirus does not replicate well in mice. Thus, demonstration of differences in the anti-tumor activity of replication-defective and replication-selective adenoviruses, as well as the potential immune system stimulation by GM-CSF production dependent upon adenoviral replication, is restricted. An immunocompetent mouse model was developed using the CMT-64 murine lung tumor cell line in C57BlI6 mice to partially circumvent this shortcoming. Low but measurable levels of replication of Ar20-1 004 (which is identical to CG0070 but encodes murine GM-CSF) and wild type Ad5 occur in this cell line in vitro.

Subcutaneous CMT-64 tumors were established in the flanks of C57Bl/6 mice, and control and test viruses were injected intratumorally three or four times at dose levels ranging from 5×1010 to 2×1010 vp with Ar20-1 004, CG0070 or saline. Measurable levels of GM-CSF in the serum and tumors were seen after intratumoral injection of tumors with Ar20-1004 or CG0070 (FIG. 2.3.3). At the high dose, Ar20-1004 showed a trend toward a greater anti-tumor effect compared to CG0070, which expresses human GM-CSF (FIG. 2.3.4).

The effect of GM-CSF in enhancing immune response was also evident in a second study, as detailed in FIG. 2.3.5. Antigen-presenting cells were enriched in tumor draining regional lymph nodes after treatment with the murine GM-CSF-expressing virus Ar20-1004. Flow cytometric analyses showed increased CD 11 c+ dendritic cells and macrophages in tumors in the Ar20-1004-treated mice compared to Ar20-1061, which is identical to Ar20-1 004 but does not encode GM-CSF.

In summary, studies in subcutaneous murine xenograft tumor models, including an orthotopic bladder TCC model, demonstrated viral replication, adenoviral cytotoxicity and the anti-tumor effects of CG0070. A study with Ar20-1004 (which is identical to CG0070 but encodes murine GM-CSF) in the CMT-64 tumor model in immunocompetent mice, which showed a trend towards enhanced tumor inhibition and increased numbers of CD11c+ cells in tumor-draining lymph nodes, suggested that GMCSF was stimulating the immune system as predicted.

### Immune Checkpoint Modulators:

1. Anti-CTLA4 Antibody:

Immune checkpoint molecules and methods of using such molecules are known and have been described in the art. An example of an immune checkpoint molecule is an anti-CTLA4 antibody. Examples of anti-CTLA-4 antibodies are ipilimumab (see U.S. Pat. Nos. 6,984,720, 7,452,535, 7,605,238, 8,017,114 and 8,142,778), tremilimuab (see U.S. Pat. Nos. 6,68,736, 7,109,003, 7,132,281, 7,411,057, 7,807,797, 7,824,679 and 8,143,379) and other anti-CTLA4 antibodies, including single chain antibodies (e.g., see U.S. Pat. Nos. 5,811,097, 6,051,227 and 7,229,628, and US Patent Publication No. US20110044953).

One of the anti-CTLA4 antibodies is now available in the clinical setting and approved by the FDA to be used in late stage melanoma patients. The complete prescribing information is fully described in the packaging insert of Yervoy™ (Bristol Meyers). Yervoy (Ipilimumab) comes in 50 mg single use vials.

Dosage: The recommended dosage ranges from 30 μg to 500 μg to be mixed into the ˜2 ml of tumor-viral vector solution, based on pre clinical data. The precise recommended dosage will be determined by the upcoming phase 1 study.

2. Anti-PD1:

Another important immune checkpoint modulator is PD1. PD-1 has been defined as a receptor for B7-4. B7-4 can inhibit immune cell activation upon binding to an inhibitory receptor on an immune cell. Agents for down modulating PD-1, B7-4, and the interaction between B7-4 and PD-1 inhibitory signal in an immune cell resulting in enhancement of the immune response and can be used in CLIVS.

U.S. Pat. No. 7,101,550
Filing date: Feb. 6, 2002
Issue date: Sep. 5, 2006
application Ser. No. 10/068,215

| Cited | Filing | Issue | Original | |
|---|---|---|---|---|
| Patent | date | date | Assignee | Title |
| U.S. Pat. No. 5,698,520 | Dec. 18, 1996 | Dec. 16, 1997 | Ono | Peptide related to human |
| | | | Pharmaceutical | programmed cell death and DNA |
| | | | Co., Ltd. | encoding the same |
| | | | Tasuku Honjo | |
| U.S. Pat. No. 6,808,710 | Aug. 23, 2000 | Oct. 26, 2004 | Genetics Institute, | Downmodulating an immune |
| | | | Inc. | response with multivalent |
| | | | Dana-Farber | antibodies to PD-1 |
| | | | Cancer Institute, | |
| | | | Inc. | |
| US20020055139 | Mar. 1, 2001 | | | Novel genes encoding proteins |
| | | | | having prognostic, diagnostic, |
| | | | | preventive, therapeutic, and |
| | | | | other uses |

### Referenced by

| Citing | Filing | Issue | | |
|---|---|---|---|---|
| Patent | date | date | Original Assignee | Title |
| U.S. Pat. No. 7,709,214 | Jun. 20, 2007 | May 4, 2010 | Dana-Farber Cancer | Methods for upregulating an |
| | | | Institute, Inc. | immune response with agents that |
| | | | Genetics Institute, | inhibit the intereaction between |
| | | | LLC | PD-L2 and PD-1 |
| U.S. Pat. No. 7,722,868 | Aug. 9, 2006 | May 25, 2010 | Dana-Farber Cancer | Agents that modulate the |
| | | | Institute, Inc. | interaction of B7-1 polypeptide |
| | | | Brigham and | with PD-L1 and methods of use |
| | | | Women's Hospital | thereof |
| U.S. Pat. No. 7,794,710 | Apr. 22, 2002 | Sep. 14, 2010 | Mayo Foundation | Methods of enhancing T cell |
| | | | for Medical | responsiveness |
| | | | Education and | |
| | | | Research | |
| U.S. Pat. No. 7,892,540 | Oct. 6, 2005 | Feb. 22, 2011 | Mayo Foundation | B7-H1 and methods of diagnosis, |
| | | | for Medical | prognosis, and treatment of cancer |
| | | | Education and | |
| | | | Research | |
| U.S. Pat. No. 7,943,743 | Jun. 30, 2006 | May 17, 2011 | Medarex, Inc. | Human monoclonal antibodies to |
| | | | | programmed death ligand 1 (PD- |
| | | | | L1) |
| U.S. Pat. No. 8,008,449 | May 2, 2006 | Aug. 30, 2011 | Medarex, Inc. | Human monoclonal antibodies to |
| | | | Ono Pharmaceutical | programmed death 1 (PD-1) and |
| | | | Co., Ltd. | methods for treating cancer using |
| | | | | anti-PD-1 antibodies alone or in |
| | | | | combination with other |
| | | | | immunotherapeutics |
| U.S. Pat. No. 8,088,905 | Mar. 16, 2009 | Jan. 3, 2012 | Wyeth | Nucleic acids encoding antibodies |
| | | | MedImmune | against PD-1 |
| | | | Limited | |
| U.S. Pat. No. 8,163,503 | May 20, 2010 | Apr. 24, 2012 | Millennium | Methods of identifying compounds |
| | | | Pharmaceuticals, | that bind TANGO509 |
| | | | Inc. | |
| U.S. Pat. No. 8,168,757 | Mar. 3, 2009 | May 1, 2012 | Merck Sharp & | PD-1 binding proteins |
| | | | Dohme Corp. | |

3. OX40

The interaction of OX40L and OX40 will sustain T cell proliferation and immune response and memory beyond the first two days. Methods for enhancing the immune response to a tumor antigen by engaging the OX-40 receptor on the surface of T-cells by a OX-40 receptor binding agent, OX40L or a OX40 agonist during or shortly after priming of the T-cells by the antigen can be used in CLIVS as an immune checkpoint modulator.

See U.S. Pat. No. 6,312,700 and the other cited and referenced patents below:

### CITATIONS

| Cited | Filing | Issue | | |
|---|---|---|---|---|
| Patent | date | date | Original Assignee | Title |
| U.S. Pat. No. 4,590,071 | Sep. 25, 1984 | May 20, 1986 | Xoma Corporation | Human melanoma specific |
| | | | | immunotoxins |
| U.S. Pat. No. 4,664,911 | Jun. 21, 1983 | May 12, 1987 | Board of Regents, | Immunotoxin conjugates |
| | | | University of Texas System | employing toxin B chain |
| | | | | moieties |
| U.S. Pat. No. 4,681,760 | Apr. 17, 1985 | Jul. 21, 1987 | The Board of Trustees of | Method of conferring |
| | | | the Leland Stanford Junior | immunotolerance to a |
| | | | University | specific antigen |
| U.S. Pat. No. 4,731,244 | Nov. 13, 1985 | Mar. 15, 1988 | Ortho Pharmaceutical | Monoclonal antibody |
| | | | Corporation | therapy |
| U.S. Pat. No. 4,867,973 | Sep. 13, 1984 | Sep. 19, 1989 | Cytogen Corporation | Antibody-therapeutic agent |
| | | | | conjugates |
| U.S. Pat. No. 5,045,451 | Oct. 26, 1988 | Sep. 3, 1991 | Board of Regents | Methods for screening |
| | | | | antibodies for use as |
| | | | | immunotoxins |
| U.S. Pat. No. 5,057,313 | Aug. 23, 1988 | Oct. 15, 1991 | The Center for Molecular | Diagnostic and therapeutic |
| | | | Medicine and Immunology | antibody conjugates |
| U.S. Pat. No. 5,057,598 | Feb. 1, 1989 | Oct. 15, 1991 | Centocor, Inc. | Monoclonal antibodies |
| | | | | reactive with endotoxin |
| | | | | core |
| U.S. Pat. No. 5,091,177 | May 11, 1990 | Feb. 25, 1992 | Oncogen | Monoclonal antibodies for |
| | | | | treatment of human non- |
| | | | | small cell lung carcinomas |
| U.S. Pat. No. 5,167,956 | Feb. 11, 1991 | Dec. 1, 1992 | The United States of | Immunotoxin with in-vivo |
| | | | America as represented by | T cell suppressant activity |
| | | | the Department of Health | |
| | | | and Human Services | |
| U.S. Pat. No. 5,329,028 | Aug. 5, 1992 | Jul. 12, 1994 | Genentech, Inc. | Carbohydrate-directed |
| | | | | cross-linking reagents |
| U.S. Pat. No. 5,376,367 | Nov. 22, 1991 | Dec. 27, 1994 | Immunex Corporation | Fusion proteins comprising |
| | | | | MGF and IL-3 |
| U.S. Pat. No. 5,457,035 | Jul. 23, 1993 | Oct. 10, 1995 | Immunex Corporation | Cytokine which is a ligand |
| | | | | for OX40 |
| U.S. Pat. No. 5,578,707 | Feb. 12, 1993 | Nov. 26, 1996 | Yeda Research and | Soluble interferon-gamma |
| | | | Development, Co., Ltd. | receptor fragment |
| U.S. Pat. No. 5,821,332 | Nov. 3, 1993 | Oct. 13, 1998 | The Board of Trustees of | Receptor on the surface of |
| | | | the Leland Stanford Junior | activated CD4.sup. + T- |
| | | | University | cells: ACT-4 |

### Referenced by

| Citing | Filing | Issue | | |
|---|---|---|---|---|
| Patent | date | date | Original Assignee | Title |
| U.S. Pat. No. 7,291,331 | Sep. 11, 2003 | Nov. 6, 2007 | La Jolla Institute | Methods of treating OX40 |
| | | | for Allergy and | medicated recall immune responses |
| | | | Immunology | |
| U.S. Pat. No. 7,531,170 | Sep. 7, 2006 | May 12, 2009 | La Jolla Institute | Methods of treating OX40 mediated |
| | | | for Allergy and | recall immune responses and agents |
| | | | Immunology | useful for identifying same |
| U.S. Pat. No. 7,550,140 | Jun. 13, 2003 | Jun. 23, 2009 | Crucell Holland | Antibody to the human OX40 |
| | | | B.V. | receptor |
| U.S. Pat. No. 7,622,444 | Sep. 29, 2006 | Nov. 24, 2009 | Sisters of | Methods for using OX-40 ligand to |
| | | | Providence in | enhance an antigen specific immune |
| | | | Oregon | response |
| U.S. Pat. No. 7,696,175 | Oct. 28, 2005 | Apr. 13, 2010 | University of | Combination cancer immunotherapy |
| | | | Southern | with co-stimulatory molecules |
| | | | California | |
| U.S. Pat. No. 7,758,852 | Apr. 2, 2003 | Jul. 20, 2010 | Merck Serono SA | OX40R binding agents |
| U.S. Pat. No. 7,807,156 | Oct. 16, 2007 | Oct. 5, 2010 | La Jolla Institute | Methods of treating OX40 mediated |
| | | | for Allergy and | recall immune responses |
| | | | Immunology | |
| U.S. Pat. No. 7,928,074 | Jun. 20, 2008 | Apr. 19, 2011 | Amgen Inc. | Combination therapy with co- |
| | | | | stimulatory factors |
| U.S. Pat. No. 7,959,925 | Nov. 13, 2009 | Jun. 14, 2011 | Providence Health | Trimeric OX40-immunoglobulin |
| | | | System | fusion protein and methods of use |
| U.S. Pat. No. 8,101,175 | Jun. 20, 2008 | Jan. 24, 2012 | La Jolla Institute | Methods of treating OX40 mediated |
| | | | for Allergy and | recall immune responses using |
| | | | Immunology | OX40L antibodies and agents useful |
| | | | | for identifying same |
| U.S. Pat. No. 8,133,983 | Mar. 23, 2009 | Mar. 13, 2012 | Crucell Holland | Agonistic binding molecules to the |
| | | | B.V. | human OX40 receptor |

4. LAG-3

The use of LAG-3 (Lymphocyte Activating Gene-3), and in a more general way, the use of MHC class II ligands or MHC class II-like ligands as adjuvants for vaccines, in order to boost an antigen specific immune response has been successful in pre clinical models. Antibodies or agents directed against or modulate LAG-3 gene products may be helpful in the present invention.

See U.S. Pat. No. 5,773,578, cited and referenced patents for details of LAG-3 related patents and claims.

### EXAMPLES

### Example 1

### For Muscle Invasive Bladder Cancer

Muscle invasive bladder as an example chosen is because CG0070 has shown to be active in bladder cancer. Furthermore all muscle invasive bladder cancer patients need to have a cystectomy, thus providing a good tumor specimen to prepare the tumor cells needed for this vaccine system. In addition the prognosis of muscle invasive bladder cancer patients (T3-4) has been quite miserable despite the use of neo-adjuvant chemotherapy. Most of these patients are over 60 years of age and few can undergo the serious side effects of chemotherapy. An effective agent that can minimize the risk of disease recurrence in this patient population is an unmet need.

### The Tumor-Viral-ICM Live and In-Vivo Cancer Vaccine System Administration:

For use as a component of the vaccine system the muscle invasive bladder tumor cells are prepared according to methods previous described, and thawed, tested for their viability, and irradiated to inactivate further cell proliferation. The first vaccination is suitably done 10 days after operation. Just prior to vaccination, 1×107 tumor cells in aliquots of about 1 ml per dose are admixed with 10× viral particles (viral particles: tumor cells=10:1) of CG0070 (in vials of 1×109 viral particles in 1 to 1.2 ml). The approximately 10× vp dose of CG0070 for the mixture will be drawn by first diluting the vial of CG0070 into 100 ml. of normal saline and then the calculated dose withdrawn from the saline bag (˜1 ml). The addition of ˜100 μg of anti-CTLA4 antibody (e.g. ipilimumab) will be the final step in the complete mixture ready for administration. The vaccination is then repeated six times in weekly intervals. This is followed by a clinical reassessment (combined with tests) of the patient according to the general guidelines for follow-up in clinical studies.

During the course of treatment changes of the immunological status of the patient during and after therapy are measured beside the general clinical data. As comparative values tests before and after operation, and before onset of immunotherapy are used. The examinations and tests comprise the cell-mediated and the humoral immune response.

With respect to application it can be summarized that it should occur not earlier than one week after surgery and no later than 4 weeks after surgery. The cancer vaccine system is to be given at least three times, preferably six or more in weekly intervals. And for testing, i.e. the determination of the DTH reaction, another injection with irradiated tumor cells with the viral vector in a lower dose (˜105 tumor cells in 0.1 ml) is administered 2 to 4 weeks after the initial course of the six weekly treatment.

The best results are obtained when autologous material, i.e. material derived from the tumor removed from the patient, is used. Alternatively, when autologous tumor cells are not available, allogeneic tumor cells derived from a third party or from bladder tumor cell lines may substitute for the autologous part of the cancer vaccine system.

For maintaining therapy, i.e. for later injections in longer intervals, e.g. every three months, which should be maintained over years, it will be recommended to use allogenic material to be mixed with CG0070 and anti-CTLA4 antibody before administration.

### Administration:

The live and in-vivo tumor-viral cancer vaccine system of ˜2 ml of a mixture of tumor cells, the oncolytic virus (CG0070) and anti-CTLA4 antibody is injected by the subcutaneous route into areas with good lymphatic drainage such as the groin area.

### Example 2

### For Castration Resistant Prostate Cancer

RB pathway defective primary prostate cancer is not common, while up to 60% of castration resistant prostate cancer may harbor such a defective pathway. These results have been confirmed through xenograft model transformation studies. Even though RB pathway defective prostate cancer may be more sensitive towards chemotherapy, there are few options left for these patients once chemotherapy fails or the patient is unsuitable for chemotherapy. Since most of these patients are elderly, an effective and less toxic agent that can delay disease progression in this patient population is an unmet need.

### The Tumor-Viral Live and In-Vivo Cancer Vaccine System Administration:

For use as a component of the vaccine system the prostate cancer tumor cells taken from biopsy specimens are prepared according to methods previous described, and thawed, tested for their viability, and irradiated to inactivate further cell proliferation. The first vaccination is suitably done any time after patients failed chemotherapy. Just prior to vaccination, 1×107 tumor cells in aliquots of about 1 ml per dose are admixed with 10× viral particles (viral particles: tumor cells=10:1) of CG0070 (in vials of 1×109 viral particles in 1 to 1.2 ml). The approximately 10× vp dose of CG0070 for the mixture will be drawn by first diluting the vial of CG0070 into 100 ml. of normal saline and then the calculated dose withdrawn from the saline bag (˜1 ml). The addition of ˜100 μg of anti-CTLA4 antibody (e.g. ipilimumab) will be the final step in the complete mixture ready for administration. The vaccination is then repeated six times in weekly intervals. This is followed by a clinical reassessment (combined with tests) of the patient according to the general guidelines for follow-up in clinical studies.

During the course of treatment changes of the immunological status of the patient during and after therapy are measured beside the general clinical data. As comparative values tests before and after operation, and before onset of immunotherapy are used. The examinations and tests comprise the cell-mediated and the humoral immune response.

The cancer vaccine system is to be given at least six to eight times, in weekly intervals. And for testing, i.e. the determination of the DTH reaction, another injection with irradiated tumor cells with the viral vector in a lower dose (˜105 tumor cells in 0.1 ml) is administered 2 to 4 weeks after the initial course of the six weekly treatment.

The best results are obtained when autologous material, i.e. material derived from the tumor removed from the patient, is used. Alternatively, when autologous tumor cells are not available, allogeneic tumor cells derived from a third party or from prostate cancer cell lines with RB defective pathway may substitute for the autologous part of the cancer vaccine system.

For maintaining therapy, i.e. for later injections in longer intervals, e.g. every three months, which should be maintained over years, it will be recommended to use allogenic material to be mixed with CG0070 and anti-CTLA4 antibody before administration.

### Administration:

The live and in-vivo tumor-viral cancer vaccine system of ˜2 ml of a mixture of tumor cells, the oncolytic virus (CG0070) and anti-CTLA4 antibody is injected by the subcutaneous route into areas with good lymphatic drainage such as the groin area.

### EQUIVALENTS

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

### REFERENCES

| Reference | Filing date | Issue date | Original Assignee | Title |
|---|---|---|---|---|
| Patent | Dec. 28, | Dec. 28, | Schirrmacher | Virus modified |
| US5273745 | 1989 | 1993 | Volker | tumor vaccine |

V0046 Clinical Study Report: Submitted to the FDA IND12154 serial number 52 in 2012. Full report will be supplemented in the non-provisional patent application
Dranoff et al: Vaccination with irradiated tumor cells with expression of murine GM-CSF. Proc. Nat. Acad. Sci. USA 1993 90 (8) 3539-3543
Ramesh: CG0070 replication competent adenovirus expressing GM-CSF: Clinical Cancer Research 2006; 12; 305-513

1. A tumor-specific immunotherapeutic vaccine system which is an apparatus comprising: at least two of the three components of:
a. separated tumor cells isolated and inactivated by irradiation,
b. an oncolytic viral and a cancer specific vector comprising a heterologous nucleic acid encoding GM-CSF and
c. an immune checkpoint modulator (“ICM”),
wherein the two or three components are admixed just prior to administration to patient without any pre-incubation.

2. The tumor-specific immunotherapeutic vaccine system of claim 1, wherein the immune checkpoint modulator is omitted.

3. The tumor-specific immunotherapeutic vaccine system of claim 1, wherein the immune checkpoint modulator is an anti-CTLA4 antibody.

4. The tumor-specific immunotherapeutic vaccine system of claim 1, wherein the anti-CTLA4 antibody is selected from ipilimumab, tremilimuab and a single chain anti-CTLA-4 antibody.

5. The tumor-specific immunotherapeutic vaccine system of claim 1, wherein the immune checkpoint modulator is an OX40 binding agent or agonist, or an OX40L molecule.

6. The tumor-specific immunotherapeutic vaccine system of claim 1, wherein the immune checkpoint modulator is an antibody or modulator selected from a group of molecules or antibodies targeted against or modulate PD1, TIM3, B7-H3, B7-H4, LAG-3 and KIR, or its ligands.

7. The tumor-specific immunotherapeutic vaccine system of claim 1, wherein the immune checkpoint modulator is a combination of two or more of an anti-CTLA4 antibody selected from the group of: an anti-CTLA4 antibody; ipilimumab, tremilimuab and a single chain anti-CTLA-4 antibody; an OX40 binding agent or agonist, or an OX40L molecule; or a group of molecules or antibodies targeted against or modulate PD1, TIM3, B7-H3, B7-H4, LAG-3 and KIR, or its ligands.

8. The tumor-specific immunotherapeutic vaccine system of claim 1, wherein the oncolytic and cancer specific viral vector expressing GM-CSF is from an adenovirus, such as CG0070.

9. The tumor-specific immunotherapeutic vaccine system of claim 1, wherein the oncolytic and cancer specific viral vector expressing GM-CSF is selected from viruses other than adenovirus, such as Herpes Simplex, Vaccinia, Mumps, Newcastle Disease and Reo viruses.

10. The tumor-specific immunotherapeutic vaccine system of claim 1, further comprising a kit comprising either at least two of three of the following components:
a. separated tumor cells isolated and inactivated by irradiation,
b. an oncolytic viral and a cancer specific vector comprising a heterologous nucleic acid encoding GM-CSF and
c. an immune checkpoint modulator (“ICM”), and a packaging insert containing directions for use.

11. The tumor-specific immunotherapeutic vaccine system of claim 1, further comprising a tumor cell preparation kit comprising: materials and methods to conduct tumor dissociation and preparation, enzymatic and/or virus vector transduction agents, cryopreservation vials etc. and a packaging insert containing directions for use.

12. The tumor-specific immunotherapeutic vaccine system of claim 1, further comprising inducing a local and/or systemic specific immune response for cancer treatment, comprising administering to the patient the tumor specific immunotherapeutic vaccine system.

13. The tumor-specific immunotherapeutic vaccine system of claim 12, further comprising, whereby the source of the cancer cells is autologous.

14. The tumor-specific immunotherapeutic vaccine system of claim 12, further comprising, whereby the source of the cancer cells is allogeneic.

15. The tumor-specific immunotherapeutic vaccine system of claim 12, further comprising, whereby the source of the cancer cells is from cell culture systems.

16. The tumor-specific immunotherapeutic vaccine system of claim 12, further comprising, whereby the cancer cells are modified to secrete or express agents or receptors that may be beneficial for viral transduction and/or immune modulation, including but not limited to, an example such as GVAX, which are modified autologous or allogeneic cancer cells secreting GM-CSF.

17. The tumor-specific immunotherapeutic vaccine system of claim 12, further comprising, whereby the source of the cancer cells can be a combination of two or more cancer cell origins as specified in claims 13 to 16.

18. The tumor-specific immunotherapeutic vaccine system of claim 12, further comprising The method of claim 12, wherein the tumor-specific immunotherapeutic vaccine system is administered to the patient subcutaneously.

19. (canceled)

20. (canceled)

21. (canceled)

22. (canceled)

23. (canceled)

24. (canceled)

25. (canceled)

26. (canceled)

27. (canceled)

28. (canceled)
